(19) United States
(12) Patent Application Publication
(10) Pub. No.: US 2010/0056815 A1
(43) Pub. Date: Mar. 4, 2010

(54) METHOD FOR PRODUCING PROPYLENE OXIDE

(75) Inventors: Tomonori Kawabata, Toyonaka-shi (JP); Hiroaki Abekawa, Toyonaka-shi (JP); Yuka Kawashita, Kobe-shi (JP)

Correspondence Address: FOLEY AND LARDNER LLP;SUITE 500, 3000 K STREET NW, WASHINGTON, DC, 20007, US

(73) Assignee: Sumitomo Chemical Company, Limited

(21) Appl. No.: 12/448,775

(22) PCT Filed: Jan. 21, 2008
(86) PCT No.: PCT/JP2008/051140

§ 371 Date: Jul. 7, 2009

(30) Foreign Application Priority Data
Jan. 24, 2007 (JP) 2007-013448

(51) Int. Cl. C07D 301/06

(52) U.S. Cl. 549/533

(57) ABSTRACT

A method for producing propylene oxide according to the present invention includes the step of reacting propylene, oxygen and hydrogen in a liquid phase in the presence of titanosilicate and a noble metal catalyst supported on a carrier comprising a noble metal catalyst and activated carbon having total pore volume of 0.9 cc/g or more. This makes it possible to provide a method for efficiently producing propylene oxide from propylene, oxygen, and hydrogen.

### TECHNICAL FIELD

The present invention relates to a method for producing propylene oxide from propylene, oxygen, and hydrogen.

### BACKGROUND ART

As a method for producing propylene oxide from propylene, oxygen, and hydrogen, for example, a method using a supported palladium compound and titanosilicate is known. As to the reaction for producing propylene oxide from hydrogen, oxygen, and propylene in a solvent containing cesium phosphate, it has been reported that the use of a catalyst in which palladium is supported on niobium oxide exhibits higher proplylene oxide productivity than a catalyst in which palladium is supported on an activated carbon (see Patent Document 1). However, the use of niobium oxide increases catalyst cost. In addition, the catalyst in which palladium is supported on niobium oxide does not always produce satisfactory reaction results.

[Patent Document 1]

Japanese Unexamined Patent Publication (Translation of PCT application) No. 2005-508362

### DISCLOSURE OF INVENTION

The present invention provides a method for efficiently producing propylene oxide from propylene, oxygen, and hydrogen.

Namely, the present invention relates to a method for producing propylene oxide which includes the step of reacting propylene, oxygen and hydrogen in a liquid phase in the presence of titanosilicate and a noble metal catalyst supported on a carrier comprising a noble metal catalyst and an activated carbon having total pore volume of 0.9 cc/g or more.

Additional objects, features, and strengths of the present invention will be made clear by the description below. Further, the advantages of the present invention will be evident from the following explanation.

### BEST MODE FOR CARRYING OUT THE INVENTION

The total pore volume of activated carbon used in the present invention is calculated by a nitrogen adsorption method at a saturation temperature of liquid nitrogen. The activated carbon used in the present invention is activated carbon having 0.9 cc/g or more total pore volume, preferably activated carbon having 1.3 cc/g or more pore volume. An upper limit to pore volume, which is however not particularly limited, usually approximately 3 cc/g. It is known that activated carbon takes various kinds of forms such as powdery form, granular form, cataclastic form, fibrous form, and honeycomb form, according to the type of its material and a producing method of activated carbon. However, activated carbon used in the present invention is not limited in forms. Examples of a raw material for activated carbon include wood, sawdust, coconut shell, coal, and petroleum. Activation is carried out by a method of processing the raw material for activated carbon with water vapor, carbon dioxide, or air at a high temperature, or a method of processing the raw material for activated carbon with a chemical such as zinc chloride. Although the present invention does not particularly impose restriction in raw material for activated carbon and activation method of the raw material, a material obtained by activation with a chemical is preferably used.

A noble metal catalyst used in the present invention is a catalyst comprising palladium compound, platinum compound, ruthenium compound, rhodium compound, iridium compound, osmium compound, gold compound, or a mixture of any of these noble metal compounds. Preferable noble metal catalyst is a noble metal catalyst comprising palladium compound, platinum compound, or gold compound. More preferred noble metal catalyst is a catalyst comprising a palladium compound.

A noble metal catalyst supported on a carrier can be prepared by having a noble metal compound which can be used as a noble metal source such as a nitrate salt of a noble metal, e.g., palladium nitrate, a sulfate salt of a noble metal, e.g., palladium sulfate dihydrate, a halogenide of a noble metal, e.g., palladium chloride, a carboxylate salt, e.g., palladium acetate, or an ammine complex, e.g., Pd tetraammine chloride or Pd tetraammine bromide, supported on an activated carbon having 0.9 cc/g or more total pore volume by an impregnation method or the like, followed by reduction with a reducing agent; or it can also be prepared by first changing a noble metal to its hydroxide with an alkali such as sodium hydroxide, followed by reduction with a reducing agent in a liquid phase or a gas phase. Examples of the reducing agent to be used in case of the reduction in a liquid phase include hydrogen, hydrazine monohydrate, formaldehyde, and sodium tetrahydroborate. When using hydrazine monohydrate or formaldehyde, the addition of an alkali is also known. Examples of the reducing agent to be used in case of the reduction in a gas phase include hydrogen and ammonia. A preferred reduction temperature is varied depending on a noble metal source supported, but generally from 0° C. to 500° C. Moreover, the catalyst can also be prepared by having an ammine complex of a noble metal, e.g., Pd tetraammine chloride or Pd tetraammine bromide supported on an activated carbon having 0.9 cc/g or more total pore volume by an impregnation method or the like, followed by reduction with ammonia gas generated upon thermal decomposition in an atmosphere of an inert gas. The reduction temperature is varied depending on an ammine complex of a noble metal, but in case of using Pd tetraammine chloride, generally from 100° C. to 500° C. and preferably 200° C. to 350° C.

In any methods, if necessary, it is possible to activate the resultant catalyst by heat treatment in an atmosphere of an inert gas, ammonia gas, vacuum, hydrogen or air. Further, after filling an oxide or hydroxide compound of a noble metal supported on an activated carbon into a reactor, it can be reduced partially or completely with hydrogen contained in starting materials of the reaction under reaction conditions. In this way, the resultant noble metal catalyst supported on a carrier generally contains a noble metal catalyst in a range of 0.01 to 20% by weight, preferably 0.1 to 5% by weight. The weight ratio of the noble metal catalyst to titanosilicate (weight of a noble metal to weight of titanosilicate) is preferably 0.01 to 100% by weight, more preferably 0.1 to 20% by weight.

Titanosilicate is a generic name of a substance in which a part of Si in a porous silicate (SiO₂) is replaced with Ti. Ti of titanosilicate is placed in SiO₂ framework, and this can be easily confirmed by a peak of 210 to 230 nm in ultraviolet-visible absorption spectra. In addition, Ti of TiO₂ is usually 6-coordination, whereas Ti of titanosilicate is 4-coordination. This can be easily confirmed by measuring coordination number in a Ti-K-edge XAFS analysis or other method.

Examples of the titanosilicate used in the present invention includes crystalline titanosilicates such as, in terms of the framework type code by IZA (International Zeolite Association), TS-2 having MEL structure, Ti-ZSM-12 having MTW structure (e.g., one described in Zeolites 15, 236-242, (1995)), Ti-Beta having BEA structure (e.g., one described in Journal of Catalysis 199, 41-47, (2001)), Ti-MWW having MWW structure (e.g., one described in Chemistry Letters 774-775, (2000)), and Ti-UTD-I having DON structure (e.g., Zeolites 15, 519-525, (1995)).

Examples of the lamellar titanosilicate include a titanosilicate having a structure with expanded interlayers in MWW structure such as Ti-MWW precursor (e.g., one described in Japanese Unexamined Patent Publication No. 2003-327425) and Ti-YNU-I (e.g. one described in Angewande Chemie International Edition 43, 236-240, (2004)).

Mesoporous titanosilicate is a generic name of titanosilicates usually having periodic pore structures of diameters ranging from 2 to 10 nm and examples thereof include Ti-MCM-41 (e.g., one described in Microporous Materials 10, 259-271, (1997)), Ti-MCM-48 (e.g., one described in Chemical Communications 145-146, (1996)), and Ti-SBA-15 (e.g., one described in Chemistry of Materials 14, 1657-1664, (2002)). Further examples of the titanosilicate include a titanosilicate having features of both mesoporous titanosilicate and titanosilicate zeolite, such as Ti-MMM-1 (e.g. one described in Microporous and Mesoporous Materials 52, 11-18, (2002)).

Among the titanosilicates used in the present invention, a crystalline titanosilicate or a lamellar titanosilicate which has pores of 12 or more membered oxygen rings is preferred. As the crystalline titanosilicate having pores of 12 or more membered oxygen rings, Ti-ZSM-12, Ti-Beta, Ti-MWW and Ti-UTD-1 are named. As the lamellar titanosilicate having pores of 12 or more membered oxygen rings, Ti-MWW precursor and Ti-YNU-I are named. As a more preferred titanosilicate, Ti-MWW and Ti-MWW precursor are named.

Usually, the titanosilicate used in the present invention can be synthesized by such a method that a surfactant is used as a template or a structure directing agent, a titanium compound and a silicon compound are hydrolyzed, if necessary, followed by improvement of crystallization or periodic regularity of pores by hydrothermal synthesis, etc., and then the surfactant is removed by calcining or extraction.

Usually, the crystalline titanosilicate having MWW structure is prepared as follows. Namely, a silicon compound and a titanium compound are hydrolyzed in the presence of a structure directing agent to prepare a gel. Then, the resultant gel is subjected to heat treatment in the presence of water, such as hydrothermal synthesis, etc. to prepare a lamellar precursor of crystal. Then, the resultant lamellar precursor of crystal is subjected to crystallization by calcination to prepare the crystalline titanosilicate having MWW structure. The titanosilicate used in the present invention includes titanosilicate silylized with a silylizing agent such as 1,1,1,3,3,3-hexamethyldisilazane, etc. Since silylization further enhances activity or selectivity, a silylized titanosilicate is also a preferred titanosilicate (for example, silylized Ti-MWW, etc.).

In addition, the titanosilicate can be used after it is activated by treatment with a hydrogen peroxide solution at an appropriate concentration. Usually, the concentration of the hydrogen peroxide solution can be in a range of 0.0001% to 50% by weight. The solvent of hydrogen peroxide solution is not particularly limited, but water or a solvent used for a propylene oxide synthesis reaction is convenient and preferable from the industrial view point. The treatment with a hydrogen peroxide solution is possible at a temperature in the range from 0 to 100° C., preferably 0 to 60° C. Usually, the time for the treatment, which depends on a hydrogen peroxide concentration, is 10 minutes to 5 hours, preferably 1 hour to 3 hours.

The reaction of the present invention is carried out in a liquid phase of water, an organic solvent, or a mixture thereof. Examples of the organic solvent include alcohols, ketones, nitrites, ethers, aliphatic hydrocarbons, aromatic hydrocarbons, halogenated hydrocarbons, esters, glycols, and a mixture thereof. Examples of the suitable organic solvent which can suppress sequentially production of by-products due to reaction with water or alcohol in a synthesis reaction of a propylene oxide compound include linear or branched saturated aliphatic nitrites and aromatic nitrites. Examples of these nitrile compounds include C2-C4 alkyl nitrile such as acetonitrile, propionitrile, isobutyronitrile and butyronitrile, and benzonitrile, with acetonitrile being preferred.

In case where a mixture of water and an organic solvent is used, usually, the ratio of water and the organic solvent is 90:10 to 0.01:99.99 by weight, preferably 50:50 to 0.01:99.99. When the ratio of water is too large, sometimes, propylene oxide is apt to react with water, which causes deterioration due to ring opening, resulting in lowering the selectivity of the propylene oxide. To the contrary, when the ratio of an organic solvent is too large, recovery costs of the solvent becomes high.

In the process of the present invention, it is also effective to add a salt selected from an ammonium salt, an alkyl ammonium salt and an alkyl aryl ammonium salt to a reaction solvent together with the titanosilicate and the noble metal catalyst supported on a carrier, because such a salt can prevent the lowering of catalyst activity or can further increase catalyst activity to enhance utilization efficiency of hydrogen. Usually, the amount of a salt selected from an ammonium salt, an alkyl ammonium salt or an alkyl aryl ammonium salt to be added is 0.001 mmol/kg to 100 mmol/kg per unit weight of solvent (in the case of a mixture of water and an organic solvent, the total weight thereof).

Examples of the salt selected from an ammonium salt, an alkyl ammonium salt and an alkyl aryl ammonium salt include a salt composed of: (1) an anion selected from sulfate ion, hydrogen sulfate ion, carbonate ion, hydrogen carbonate ion, phosphate ion, hydrogen phosphate ion, dihydrogen phosphate ion, hydrogen pyrophosphate ion, pyrophosphate ion, halogen ion, nitrate ion, hydroxide ion, and C1-C10 carboxylate ion; and (2) a cation selected from ammonium, alkyl ammonium, and alkyl aryl ammonium.

Examples of the C1-C10 carboxylate ion include formate ion, acetate ion, propionate ion, butyrate ion, valerate ion, caproate ion, caprylate ion, and caprinate ion. Examples of the alkyl ammonium include tetramethylammonium, tetraethylammonium, tetra-n-propylammonium, tetra-n-butylammonium, and cetyltrimethylammonium.

Preferred examples of the salt selected from an ammonium salt, an alkyl ammonium salt or an alkyl aryl ammonium salt include ammonium salts of inorganic acids such as ammonium sulfate, ammonium hydrogen sulfate, ammonium carbonate, ammonium hydrogen carbonate, diammonium hydrogen phosphate, ammonium dihydrogen phosphate, ammonium phosphate, ammonium hydrogen pyrophosphate, ammonium pyrophosphate, ammonium chloride, and ammonium nitrate; or ammonium salts of C1 to C10 carboxylic acids such as ammonium acetate, and a preferred ammonium salt is ammonium dihydrogen phosphate.

In the method of the present invention, the addition of quinoid compound to a reaction solvent together with titano silicate and a noble metal catalyst supported on a carrier is also effective because it enables selectivity of propylene oxide to be greater.

Examples of the quinoid compound include a phenanthraquinone compound and a ρ-quinoid compound represented by the formula (1):

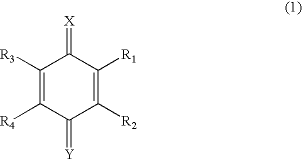

wherein R₁, R₂, R₃ and R₄ represent a hydrogen atom, adjacent pairs of R₁ and R₂, and R₃ and R₄ each are independently bonded to each other at their terminal ends and form a benzene ring optionally substituted with an alkyl group or a hydroxyl group, or a naphthalene ring optionally substituted with an alkyl group or a hydroxyl group, together with carbon atoms of quinone to which R₁, R₂, R₃ and R₄ are bonded, and X and Y are the same or different and represent an oxygen atom or a NH group.

Examples of the compound represented by the formula (1) include (1) a quinone compound (IA): the compound represented by the formula (1), wherein R₁, R₂, R₃ and R₄ are hydrogen atoms, and both X and Y are oxygen atoms; (2) a quinone-imine compound (IB): the compound represented by the formula (1), wherein R₁, R₂, R₃ and R₄ are hydrogen atoms, X is an oxygen atom, and Y is a NH group; and (3) a quinone-diimine compound (1C): the compound represented by the formula (1), wherein R₁, R₂, R₃ and R₄ are hydrogen atoms, and both X and Y are NH groups.

The quinoid compound represented by the formula (1) includes an anthraquinone compound represented by the formula (2):

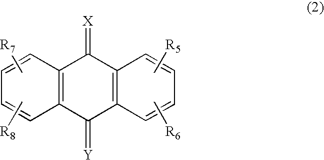

wherein X and Y are as defined in the formula (1), and R₅, R₆, R₇ and R₈ are the same or different and represent a hydrogen atom, a hydroxyl group, or an alkyl group (e.g., C1-C5 alkyl such as methyl, ethyl, propyl, butyl, and pentyl).

In the formula (1) and formula (2), X and Y preferably represent an oxygen atom. The quinoid compound represented by the formula (1) wherein X and Y are an oxygen atom is particularly referred to as quinone compound or ρ-quinone compound, and the quinoid compound represented by the formula (2) wherein X and Y are an oxygen atom is particularly referred to as anthraquinone compound.

Examples of the dihydro-form of the quinoid compound include dihydro-forms of the compounds represented by the foregoing formulas (1) and (2), i.e. compounds represented by the formulas (3) and (4):

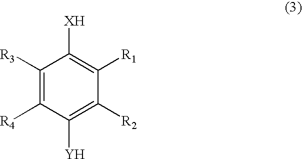

wherein R₁, R₂, R₃, R₄, X and Y are as defined in the foregoing formula (1); and

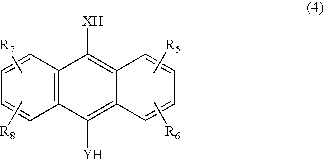

wherein X, Y, R₅, R₆, R₇ and R₈ are as defined in the foregoing formula (2).

In the formula (3) and formula (4), X and Y preferably represent an oxygen atom. The dihydro-form of quinoid compound represented by the formula (3) wherein X and Y are an oxygen atom is particularly referred to as dihydroquinone compound or dihydro ρ-quinone compound, and the dihydro-form of quinoid compound represented by the formula (4) wherein X and Y are an oxygen atom is particularly referred to as dihydroanthraquinone compound.

Examples of the phenanthraquinone compound include 1,4-phenanthraquinone as a ρ-quinoid compound and 1,2-, 3,4-, and 9,10-phenanthraquinone as o-quinoid compounds.

Specific examples of the quinone compound include: benzoquinone; naphthoquinone; anthraquinone; 2-alkylanthraquinone compounds such as 2-ethylanthraquinone, 2-t-butylanthraquinone, 2-amylanthraquinone, 2-methylanthraquinone, 2-butylanthraquinone, 2-t-amylanthraquinone, 2-isopropylanthraquinone, 2-s-butylanthraquinone and 2-s-amylanthraquinone; 2-hydroxyanthraquinone; polyalkylanthraquinone compounds such as 1,3-diethylanthraquinone, 2,3-dimethylanthraquinone, 1,4-dimethylanthraquinone and 2,7-dimethylanthraquinone; polyhydroxyanthraquinone such as 2,6-dihydroxyanthraquinone; naphthoquinone; and a mixture thereof.

Preferred examples of the quinoid compound include anthraquinone, and 2-alkylanthraquinone compounds (in formula (2), X and Y are an oxygen atom, R₅ is an alkyl group substituted at 2 position, R₆ represents a hydrogen atom, and R₇ and R₈ represent a hydrogen atom). Preferred examples of the dihydro-form of quinoid compound include the corresponding dihydro-forms of these preferred quinoid compounds.

The addition of the quinoid compound or the dihydro-form of quinoid compound (hereinafter, abbreviated as the quinoid compound derivative) to a reaction solvent can be carried out by first dissolving the quinoid compound derivative in a liquid phase and then subjecting it to the reaction. For example, a hydride compound of the quinoid compound such as hydroquinone or 9,10-anthracenediol may be added to a liquid phase, followed by oxidation with oxygen in a reactor to generate the quinoid compound and use it in the reaction.

Further, the quinoid compounds used in the present invention including the quinoid compounds exemplified above may become dihydro-forms of partly hydrogenated quinoid compounds depending on reaction conditions, and these compounds may also be used.

Usually, the amount of the quinoid compound to be used per unit weight of a solvent (unit weight of water, an organic solvent or a mixture thereof) can be in a range of 0.001 mmol/kg to 500 mmol/kg. A preferred amount of the quinoid compound is 0.01 mmol/kg to 50 mmol/kg.

In the method of the present invention, it is possible to add (a) a quinoid compound and (b) a salt selected from an ammonium salt, an alkyl ammonium salt, and an alkyl aryl ammonium salt to a reaction system a the same time.

Examples of the reaction in the present invention include a fixed bed reaction, an agitating tank type reaction, a fluidized bed reaction, a moving bed reaction, a bubble column type reaction, a tubular type reaction, and a circulating reaction. Usually, the partial pressure ratio of oxygen and hydrogen fed to a reactor is in a range of 1:50 to 50:1. A preferable partial pressure ratio of oxygen and hydrogen is 1:2 to 10:1. When the partial pressure ratio of oxygen and hydrogen (oxygen/hydrogen) is too high, the production rate of propylene oxide can be lowered. On the other hand, when the partial pressure ratio of oxygen and hydrogen (oxygen/hydrogen) is too low, selectivity of propylene oxide can be lowered due to the increase in paraffin by-products. Oxygen and hydrogen gases used in the present reaction can be used by diluting them with a gas for dilution. Examples of the gas for dilution include nitrogen, argon, carbon dioxide, methane, ethane and propane. Although the concentration of the gas for dilution is not particularly limited, the reaction is carried out by diluting oxygen or hydrogen, where necessary.

Examples of the oxygen source include oxygen gas or air. The oxygen gas can be an inexpensive oxygen gas produced by a pressure swing method, or if necessary, a high purity oxygen gas produced by cryogenic separation or the like.

Usually, the reaction temperature in the present reaction is in the rage from 0° C. to 150° C., preferably 40° C. to 90° C. When the reaction temperature is too low, the reaction rate becomes slow. On the other hand, when the reaction temperature is too high, by-products increase due to side reactions.

The reaction pressure is not particularly limited, and generally in the range from 0.1 MPa to 20 MPa in gauge pressure, preferably 1 MPa to 10 MPa. When the reaction pressure is too low, dissolution of raw material gases becomes insufficient, and the reaction rate becomes slow. When the reaction pressure is too high, costs of reaction facilities increase. Recovery of the product of the present invention, i.e., the resulting propylene oxide can be carried out by conventional distillation separation. Unreacted propylene and/or solvent(s) can also be recovered, for example, by distillation separation or membrane filtration, if necessary.

### EXAMPLES

The present invention will be explained with reference to Examples below, but the present invention is not limited thereto.

### Example 1

Ti-MWW used in this reaction was prepared by a method described in Chemistry Letters 774-775, (2000). 9.1 kg of Piperidine, 25.6 kg of purified water, 6.2 kg of boric acid, 0.54 kg of TBOT (tetra-n-butylorthotitanate) and 4.5 kg of fumed silica (cab-o-sil M7D) were placed in an autoclave and stirred at room temperature under an argon atmosphere to prepare a gel. The gel was aged for 1.5 hours, and the autoclave was closed. After the temperature was raised over 10 hours with stirring, it was maintained at 170° C. for 168 hours to conduct hydrothermal synthesis, thereby obtaining a suspension. The resultant suspension was filtered, and then washed with water until the filtrate became about pH 10. Then, the filter cake was dried at 50° C. to obtain a white powder still in a wet state. To 350 g of the resultant powder was added 3.5 L of 13% by weight nitric acid was added, and the mixture was refluxed for 20 hours. Then, the mixture was filtered, washed with water until it became approximately neutral, and dried sufficiently at 50° C. to obtain 98 g of a white powder. This white powder was subjected to X-ray diffraction pattern measurement by using an X-ray diffraction apparatus using copper K-alpha radiation. As a result, Ti-MWW precursor was confirmed. The resultant Ti-MWW precursor was calcined at 530° C. for 6 hours to obtain a Ti-MWW catalyst powder. It was confirmed that the resultant powder had MWW structure by measuring X-ray diffraction pattern, and the content of titanium by ICP emission analysis was 0.9% by weight. By using 100 g of a solution of water/acetonitrile=20/80 (weight ratio) containing 0.1% by weight of hydrogen peroxide, 0.6 g of Ti-MWW powder obtained in Example 1 was treated at room temperature for 1 hour, and the mixture was filtered and washed with 500 mL of water. The resulting Ti-MWW treated with hydrogen peroxide was used in the reaction.

The noble metal catalyst supported on a carrier used in this reaction was prepared by the following method. Note that the total pore volume of activated carbon can be measured in the manner below by using Autosorb-6 (QUANTACHROME)(or an apparatus having functions equivalent to Autosorb-6). More specifically, the total pore volume was calculated from the amount of nitrogen gas adsorption at a relative pressure of about 0.99 on an adsorption isotherm obtained by having nitrogen gas adsorbed into a sample, which was dried in advance in a vacuum at 150° C. for 4 hours, at a liquid nitrogen temperature. In a 500 mL-flask, 300 mL of aqueous solution containing 0.30 mmol of Pd tetraammine chloride was prepared. To the aqueous solution was added 3 g of commercial AC (active carbon in powdery form; pore volume: 1.57 cc/g; Wako Pure Chemical Industries, Ltd.), and the resulting mixture was stirred for 8 hours. After completion of stirring, water was removed with a rotary evaporator, and the residue was further dried at 50° C. for 12 hours under vacuum. The resultant catalyst precursor powder was calcined at 300° C. for 6 hours under a nitrogen atmosphere to obtain a noble metal catalyst supported on a carrier.

An autoclave of 0.5 L capacity was used as a reactor in the reaction. To the reactor were fed raw material gases of propylene/oxygen/hydrogen/nitrogen having a ratio of 4/8/1/87 (volume ratio) at a rate of 16 L per hour and a solution of water/acetonitrile=20/80 (weight ratio) at a rate of 108 mL per hour, while the reaction mixture was took out through a filter from the reactor, thereby conducting a continuous reaction under conditions of temperature at 60° C., pressure at 0.8 MPa (gauge pressure) and retention time of 90 minutes. During this time, 131 g of the reaction solvent, 0.133 g of Ti-MWW treated with hydrogen peroxide and 0.03 g of Pd/AC were present in the reaction mixture in the reactor. The liquid and gas phases were taken out 5 hours after the initiation of the reaction and were analyzed by gas chromatography. As a result, the activity of propylene oxide generation relative to the unit weight of Ti-MWW was 24.1 mmol-PO/g-Ti-MWW·h, selectivity based on propylene was 86% and selectivity based on hydrogen was 35%.

### Example 2

The operation was carried out in a similar manner as in EXAMPLE 1 except that commercial AC (Carborafin-6; pore volume: 1.84 cc/g; Japan EnviroChemicals, Ltd.) was used in place of the AC (active carbon in powdery form; Wako Pure Chemical Industries, Ltd.). The liquid and gas phases were taken out 5 hours after the initiation of the reaction and were analyzed by gas chromatography. As a result, the activity of propylene oxide generation relative to the unit weight of Ti-MWW was 21.0 mmol-PO/g-Ti-MWW·h, selectivity based on propylene was 76% and selectivity based on hydrogen was 27%.

### Comparative Example 1

The operation was carried out in a similar manner as in EXAMPLE 1 except that commercial AC (Yashicoal-LL; pore volume: 0.47 cc/g; Taihei Kagaku Sangyo Co., Ltd.) was used in place of the AC (active carbon in powdery form; Wako Pure Chemical Industries, Ltd.). The liquid and gas phases were taken out 5 hours after the initiation of the reaction and were analyzed by gas chromatography. As a result, the activity of propylene oxide generation relative to the unit weight of Ti-MWW was 12.0 mmol-PO/g-Ti-MWW·h, selectivity based on propylene was 65% and selectivity based on hydrogen was 24%.

### Comparative Example 2

The operation was carried out in a similar manner as in EXAMPLE 1 except that commercial niobic acid (CBMM) was used in place of the AC (active carbon in powdery form; Wako Pure Chemical Industries, Ltd.). The liquid and gas phases were taken out 5 hours after the initiation of the reaction and were analyzed by gas chromatography. As a result, the activity of propylene oxide generation relative to the unit weight of Ti-MWW was 12.7 mmol-PO/g-Ti-MWW·h, selectivity based on propylene was 92% and selectivity based on hydrogen was 38%.

TABLE 1
Results of epoxidation

| | | | PO | PO |
|---|---|---|---|---|
| | Pore | Activity | selectivity | selectivity |
| | volume | (mmol-PO/ | (% based on | (% based on |
| | (cc/g) | g-cat · h) | propylene) | H₂) |
| Example 1 | 1.57 | 24.1 | 86 | 35 |
| Example 2 | 1.84 | 21.0 | 76 | 27 |
| Comparative | 0.47 | 12.0 | 65 | 24 |
| Example 1 | | | | |
| Comparative | — | 12.7 | 92 | 38 |
| Example 2 | | | | |

### Example 3

The operation was carried out in a similar manner as in EXAMPLE 1 except that commercial AC (Carborafin-6; pore volume: 1.84 cc/g; Japan EnviroChemicals, Ltd.) was used in place of the AC (active carbon in powdery form; Wako Pure Chemical Industries, Ltd.) and the solution of water/acetonitrile=20/80 containing anthraquinone of 0.7 mmol/kg and ammonium dihydrogen phosphate of 0.7 mmol/kg was used in place of a solution of water/acetonitrile=20/80 (weight ratio). The liquid and gas phases were taken out 5 hours after the initiation of the reaction and were analyzed by gas chromatography. As a result, the activity of propylene oxide generation relative to the unit weight of Ti-MWW was 25.0 mmol-PO/g-Ti-MWW·h, selectivity based on propylene was 95% and selectivity based on hydrogen was 49%.

### Example 4

The operation was carried out in a similar manner as in EXAMPLE 1 except that commercial AC (Ryujo Shirasagi GC-100; pore volume: 0.93 cc/g; Japan EnviroChemicals, Ltd.) was used in place of the AC (active carbon in powdery form; Wako Pure Chemical Industries, Ltd.) and the solution of water/acetonitrile=20/80 containing anthraquinone of 0.7 mmol/kg and ammonium dihydrogen phosphate of 0.7 mmol/kg was used in place of a solution of water/acetonitrile=20/80 (weight ratio). The liquid and gas phases were taken out 5 hours after the initiation of the reaction and were analyzed by gas chromatography. As a result, the activity of propylene oxide generation relative to the unit weight of Ti-MWW was 11.5 mmol-PO/g-Ti-MWW·h, selectivity based on propylene was 93% and selectivity based on hydrogen was 51%.

### Comparative Example 3

The operation was carried out in a similar manner as in EXAMPLE 1 except that commercial AC (Shirasagi-M; Lot: M480; pore volume: 0.70 cc/g; Japan EnviroChemicals, Ltd.) was used in place of the AC (active carbon in powdery form; Wako Pure Chemical Industries, Ltd.) and the solution of water/acetonitrile=20/80 containing anthraquinone of 0.7 mmol/kg and ammonium dihydrogen phosphate of 0.7 mmol/kg was used in place of a solution of water/acetonitrile=20/80 (weight ratio). The liquid and gas phases were taken out 5 hours after the initiation of the reaction and were analyzed by gas chromatography. As a result, the activity of propylene oxide generation relative to the unit weight of Ti-MWW was 8.9 mmol-PO/g-Ti-MWW·h, selectivity based on propylene was 76% and selectivity based on hydrogen was 21%.

### Comparative Example 4

The operation was carried out in a similar manner as in EXAMPLE 1 except that commercial niobic acid (CBMM) was used in place of the AC (active carbon in powdery form; Wako Pure Chemical Industries, Ltd.) and the solution of water/acetonitrile=20/80 containing anthraquinone of 0.7 mmol/kg and ammonium dihydrogen phosphate of 0.7 mmol/kg was used in place of a solution of water/acetonitrile=20/80 (weight ratio). The liquid and gas phases were taken out 5 hours after the initiation of the reaction and were analyzed by gas chromatography. As a result, the activity of propylene oxide generation relative to the unit weight of Ti-MWW was 9.3 mmol-PO/g-Ti-MWW·h, selectivity based on propylene was 96% and selectivity based on hydrogen was 58%.

TABLE 2
Results of epoxidation

| | | | PO | PO |
|---|---|---|---|---|
| | Pore | Activity | selectivity | selectivity |
| | volume | (mmol-PO/ | (% based on | (% based on |
| | (cc/g) | g-cat · h) | propylene) | H₂) |
| Example 3 | 1.84 | 25.0 | 95 | 49 |
| Example 4 | 0.93 | 11.5 | 93 | 51 |
| Comparative | 0.7 | 8.9 | 76 | 21 |
| Example 3 | | | | |
| Comparative | — | 9.3 | 96 | 58 |
| Example 4 | | | | |

According to the present invention, it is possible to efficiently produce propylene oxide from propylene, oxygen, and hydrogen in the presence of titano silicate and a noble metal catalyst supported on a carrier comprising inexpensive activated carbon as a carrier.

Specific embodiments or examples implemented in the description of the embodiments only show technical features of the present invention and are not intended to limit the scope of the invention. Variations can be effected within the spirit of the present invention and the scope of the following claims.

### INDUSTRIAL APPLICABILITY

The present invention enables efficient production of propylene oxide from propylene, oxygen, and hydrogen.

1. A method for producing propylene oxide, comprising the step of:
reacting propylene, oxygen and hydrogen in a liquid phase in the presence of titanosilicate and a noble metal catalyst supported on a carrier comprising a noble metal catalyst and activated carbon having total pore volume of 0.9 cc/g or more.

2. The method according to claim 1, wherein
the activated carbon has total pore volume of 1.3 cc/g or more.

3. The method according to claim 1, wherein
the titanosilicate is crystalline titanosilicate having an MWW structure or lamellar titanosilicate.

4. The method according to claim 1, wherein
the titanosilicate is a crystalline titanosilicate having an MWW structure or a Ti-MWW precursor.

5. The method according to claim 1, wherein
the noble metal catalyst is a palladium compound, platinum compound, ruthenium compound, rhodium compound, iridium compound, osmium compound, gold compound, or a mixture of any of these compounds.

6. The method according to claim 5, wherein
the noble metal catalyst is a palladium compound.

7. The method according to claim 1, wherein
the liquid phase contains an organic solvent.

8. The method according to claim 7, wherein
the organic solvent is an organic solvent selected from alcohol, ketone, nitrile, ether, aliphatic hydrocarbon, aromatic hydrocarbon, halogenated hydrocarbon, ester, glycol, and a mixture of any of these substances.

9. The method according to claim 7, wherein
the organic solvent is acetonitrile.

10. The method according to claim 7, wherein
the liquid phase is a mixture of an organic solvent and water, and the ratio of an organic solvent and water is 90:10 to 0.01:99.99.

11. The method according to claim 7, wherein
the liquid phase contains a salt selected from an ammonium salt, an alkyl ammonium salt, and an alkyl aryl ammonium salt.

12. The method according to claim 11, wherein
the salt selected from an ammonium salt, an alkyl ammonium salt, and an alkyl aryl ammonium salt is a salt composed of (1) an anion selected from, sulfate ion, hydrogen sulfate ion, carbonate ion, hydrogen carbonate ion, phosphate ion, hydrogen phosphate ion, dihydrogen phosphate ion, hydrogen pyrophosphate ion, pyrophosphate ion, halogen ion, nitrate ion, hydroxide ion, and C1-C10 carboxylate ion; and (2) a cation selected from ammonium, alkyl ammonium, and alkyl aryl ammonium.

13. The method according to claim 11, wherein
the ammonium salt is a salt composed of ammonium cation.

14. The method according to claim 11, wherein
the ammonium salt is ammonium dihydrogen phosphate.

15. The method according to claim 7, wherein
the liquid phase contains a quinoid compound or a dihydro-form of quinoid compound.

16. The method according to claim 15, wherein the quinoid compound is a phenanthraquinone compound or a compound represented by the formula (1):
wherein R₁, R₂, R₃ and R₄ represent a hydrogen atom, adjacent pairs of R₁ and R₂, and R₃ and 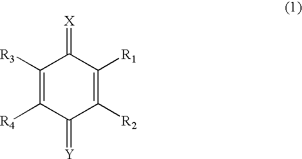
R₄ each are independently bonded to each other at their terminal ends and form a benzene ring optionally substituted with an alkyl group or a hydroxyl group, or a naphthalene ring optionally substituted with an alkyl group or a hydroxyl group, together with carbon atoms of quinone to which R₁, R₂, R₃ and R₄ are bonded, and X and Y are the same or different and represent an oxygen atom or a NH group.

17. The method according to claim 15, wherein the quinoid compound is a phenanthraquinone compound or a compound represented by the formula (2): 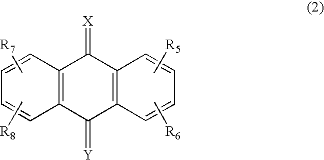
wherein X and Y are the same or different and represent an oxygen atom or a NH group, and R₅, R₆, R₇, and R₈ are the same or different and represent a hydrogen atom, a hydroxyl group, or an alkyl group.

18. The method according to claim 16, wherein
both X and Y are oxygen atoms.

19. The method according to claim 17, wherein
both X and Y are oxygen atoms.
